(19) United States
(12) Patent Application Publication
(10) Pub. No.: US 2023/0270448 A1
(43) Pub. Date: Aug. 31, 2023

(54) AUTOMATED EMERGENCY PNEUMATIC TOURNIQUET

(71) Applicant: GOLDEN HOUR MEDICAL, LLC, Boca Raton, FL (US)

(72) Inventors: Hannah D. Herbst, Boca Raton, FL (US); Devin R. Willis, Boca Raton, FL (US)

(73) Assignee: GOLDEN HOUR MEDICAL, LLC, Boca Raton, FL (US)

(21) Appl. No.: 18/107,925

(22) Filed: Feb. 9, 2023

Related U.S. Application Data
(60) Provisional application No. 63/308,267, filed on Feb. 9, 2022.

(51) Int. Cl. A61B 17/135

(52) U.S. Cl. CPC A61B 17/1355; A61B 2017/12004

(57) ABSTRACT

An automated emergency pneumatic tourniquet system including multiple interchangeable tourniquet cuffs, each having an inflatable bladder and an indicator tag designating the type of cuff, preferred pressure of application, and other data. A detachable controller reads the indicator tag and inflates the bladder to a predetermined pressure, which can be modified once applied. The detachable controller may be used to inflate multiple cuffs individually and monitor the amount the pressure applied by the cuffs. It also allows for periodic reperfusion for reducing and/or preventing ischemia. The cuff is held in place on a limb using a manual tightening mechanism independent of the bladder and controller.

### CROSS-REFERENCE TO RELATED APPLICATIONS

This application claims priority to U.S. Provisional Application Ser. No. 63/308,267 filed on Feb. 9, 2022.

### STATEMENT REGARDING FEDERALLY SPONSORED RESEARCH OR DEVELOPMENT

Not Applicable.

### NAMES OF PARTIES TO A JOINT RESEARCH AGREEMENT

Not Applicable

### REFERENCE TO SEQUENCE LISTING, A TABLE, OR A COMPUTER PROGRAM LISTING APPENDIX SUBMITTED ON A COMPACT DISC AND INCORPORATION-BY-REFERENCE OF THE MATERIAL

Not Applicable.

### BACKGROUND OF THE INVENTION

### Field of the Invention

The present invention relates to emergency tourniquets, and, more particularly, relates to a tourniquet system having an electronic tourniquet controller that may be used with several separate tourniquets on different persons, and which provides detailed instructions to an operator.

### Description of the Related Art

Tourniquets are the most common emergency medical devices for preventing death from blood loss. Tourniquets prevent exsanguination from extremity hemorrhage following trauma by applying high amounts of pressure proximal to the point of injury. The resulting compression of arteries and veins cuts off the blood supply to the limb, which prevents mortality but is strongly associated with morbidity. There are many difficulties in properly utilizing tourniquets in emergency settings.

Emergency tourniquets, invented in 200 B.C., are applied to the arm or leg to stop uncontrolled extremity hemorrhage. The first “stick-and-cloth” tourniquets were composed of a windlass “stick” tied into a “cloth” wrapped around the circumference of the limb proximal to the point of injury. The stick was repeatedly wound 360 degrees until enough circumferential pressure was achieved to visually stop the bleed. Emergency tourniquets of the prior art incorporate a windlass mechanism visually and functionally similar to the stick-and-cloth model.

Windlass tourniquets are limited in that they require training for proper use. One study of 100 medical students found that 38% of students failed to properly apply a windlass tourniquet after reading the included instructions. Two thirds of civilian laypersons who self-reported first aid training failed to correctly apply a windlass tourniquet in a study of 317 people. In another study of 198 untrained people, only 16% could apply a tourniquet properly.

Furthermore, a need exists to better treat pediatric casualties: there were more than 300 shooting incidents on K-12 school properties in 2022, resulting in 332 casualties and fatalities. 19 of the 21 people killed during the Ulvade elementary school shooting were children between the ages of 9 and 11. Thus there is an urgent need for an intuitive, automated emergency tourniquet that eliminates the need for training and is effective on pediatric limbs.

The United States Department of Defense predicts that multi-domain operations (MDO) of the future will mirror casualty volumes and medical intervention times similar to those of WWI and WWII, which can leave wounded warfighters stranded for up to 72 hours. Exsanguination and combat wound infection are the leading causes of death on the battlefield, with over 90% of combat deaths caused by exsanguinating hemorrhage. Current standard issue tourniquets for treating extremity hemorrhage are not useful for preventing infection or maintaining limb function in PC scenarios. It is impossible to administer antibiotics below the tourniquet line due to lack of circulation and tissue oxygenation, which makes patients more susceptible to local and systemic infection. Additionally, mass casualty civilian incidents may require tourniquet application that exceeds two hours due to a high volume of patients, such as in the Boston Marathon Bombing or recent mass shootings.

Windlass emergency tourniquets have proven useful for decreasing mortality in military and civilian casualty scenarios when trained personnel are available to apply them, however they are strongly associated with morbidity.

Ischemia is associated with tourniquet use, as it occurs when blood supply to tissue is inadequate. Ischemia-reperfusion injury (IRI) occurs after restoring blood flow to hypoxic or anoxic tissue, and is associated with serious complications well established in literature. Prolonged periods of ischemia results in metabolic disorders such as decreased levels of glucose and pyruvate, as well as the accumulation of lactate and glycerol.

Permanent nerve injury can occur after just two hours of tourniquet use, and permanent muscle damage is nearly complete after six hours with likely amputation. This is due to the application of a high amount of pressure concentrated over a narrow surface area (<10.5 cm tourniquet width), which has been shown in literature to result in neurophysiological damage following windlass tourniquet application. This high pressure, which is known in literature to exceed 500 mmHg, results in an axial force applied along the underlying soft tissue and nerves, which expedites nerve damage by compressing the myelin sheath surrounding nerve cells. This leads to complications which increase over time, including neuropraxia, nerve paralysis, rhabdomyolysis, compartment syndrome, increased intravascular coagulation, which is unhelpful for the PC scenarios anticipated for next generation warfighting and civilian casualty incidents. The engagement of just one windlass tourniquet has also shown to be inadequate in stopping leg bleeds in the majority of patients. Two tourniquets are applied as a standard among medics for minimum effectiveness, both of which are of exceedingly high pressure.

Reperfusion during tourniquet use is not standardized across literature and has been the subject of much debate. If the ischemic period in tourniquet application prior to medical intervention is reduced or dispersed through a combination of personalized tourniquet pressure and periodic reperfusion, then the severity of IRI will be reduced and tissue oxygenation will be preserved.

Pneumatic tourniquets are composed of an inflatable single or dual bladder that applies circumferential pressure to a limb to stop blood flow past the tourniquet (TQ). Automated pneumatic tourniquets of the prior art are nearly exclusively described for use in surgical applications to create a bloodless field, as they are cost-prohibitive for emergency use, require a large power supply, and are not portable. Other pneumatic tourniquets are utilized for venipuncture or blood sampling applications.

The use of the lowest possible pressure to occlude blood flow at the TQ is desirable to prevent damage to the underlying nerves and tissue. However, ideal pneumatic tourniquet inflation pressures are not standardized across literature. The most common methods in prior art for determining low occlusion pressures are the estimation of a Limb Occlusion Pressure (LOP) or identification of blood pressure through the oscillometric method.

Limb Occlusion Pressure (LOP) is determined prior to a surgical procedure by inflating the tourniquet cuff to cessation of a distal pulse as determined by a Photoplethysmographic (PPG) sensor on a digit distal to the TQ, or an acoustic disappearance of cardiac auscultation detected by a Doppler Ultrasound distal to tourniquet placement. After the disappearance of a cardiac signal, the physician calculates limb occlusion pressure manually or by relying on a standard from literature.

LOP is determined in many pneumatic tourniquet systems using a distal PPG sensor. Ascending LOP is calculated by inflating the tourniquet cuff until the distal pulse is lost, while descending LOP is calculated by deflating the tourniquet cuff from a very high pressure until a pulse is detected, and then adding a safety margin above the occlusion pressure. In many prior art systems, PPG is located downstream of the pneumatic TQ to determine LOP.

PPG sensors apply light to a patient's skin via light emitting diodes (LEDs) and measure the frequency of reflected light using a photodiode either adjacent to the light (reflection) or on the opposite side of the skin (transmission). Typically applied to the wrist, finger, toe, or earlobe, PPG sensors utilize light to quantify heart rate and pulse oximetry depending on the volume of blood present with each heartbeat. Despite widespread use, PPG sensors have been found to only detect 20% of the pulsatile flow within the finger in surgical tourniquet applications. Once 20% of the blood flow is occluded, the PPG sensor will relay that no heartbeat is detected to the user, which is inaccurate even for controlled environments. Although used widely in operating rooms, they are impractical for use in field or emergency environments where there is no guarantee that fingers or toes distal to the TQ are intact. Additionally, the need to apply a distal sensor while treating a casualty is not conducive to the speed and practicality necessary for treatment in emergency scenarios. Moreover, PPG sensors integrated into the TQ have been found by literature to be unreliable for emergency tourniquet field use as it requires skin contact and therefore cannot be used over clothing, and is inconsistent on muscle-dense patients, obese patients, and patients with dark skin tones.

Auscultatory doppler techniques have been employed in several surgical pneumatic systems of the prior art for detecting LOP as an alternative to PPG. Doppler techniques require large power supplies, distal placement to the tourniquet, and a quiet setting for accurate reading, and therefore are impractical for emergency application.

Surgical pneumatic tourniquet systems of the prior art also incorporate dual-purpose cuffs, for both occluding blood flow past the TQ and for identifying the limb occlusion pressure (LOP). They are typically based on the identification of a patient's systolic blood pressure through oscillation in combination with one of the aforementioned sensors (PPG, auscultatory doppler, manual stethoscope, and the like). Although accurate, blood pressure cuffs require the use of a strict measuring protocol which is time-consuming and tedious for physicians and certainly not practical for field use.

Other documents describe a limb occlusion device that has a dual-cuff system. One cuff is to be held at a high enough pressure to stop the flow of blood through the limb, while the other is to recognize the presence of an arterial blood flow through oscillations. Although a mentioned application is toward emergency use in the field, consideration is not given to limitations of using the device in the field: patient movement is virtually guaranteed following the onset of a tourniquet application which makes accurate oscillatory blood pressure measurement impossible, therefore the basis for tightening above a limb occlusion pressure is erroneous. A single battery is not enough to power the device for long enough especially for prolonged care scenarios where application time can exceed 72 hours, and consideration is not given to the type of battery which may be impacted by extreme temperatures.

Blood pressure cuffs also incorporate an inflator and an inflatable member, however the inflatable member is purposed to apply a select amount of pressure to the brachial or radial artery for the purpose of identifying a biological signal and recording the pressure at which the signal was identified, rather than fully occluding the arterial and/or venous flow to prevent exsanguination.

Pneumatic systems have however been incorporated into some emergency tourniquet systems of the prior art. The prior art discloses a device that incorporates a “slap-band” like application method, inflatable member, and CO₂ canister containing pressurized gas, where the tourniquet is applied and compressed air is released slowly into the inflatable member using a valve. This device is limited in that compressed air is a limited source of air, especially for prolonged care scenarios. Additionally, the mechanical nature of this device makes it prone to error and does not incorporate safeties. There is an indication that a valve may be employed for controlled air release, however the incorporation of a manual pressure gauge is not conducive to portability or emergency use.

One pneumatic Emergency and Military Tourniquet (EMT) includes an inflation bulb for pushing air into an inflatable member to stop the bleed. This tourniquet includes a wide cuff which is purposed to reduce inflation pressures, however the inflation pressure is not indicated to the patient. This easily allows the device to be over or under inflated, which is associated with complications. Additionally, the device is unintuitive and tedious, requiring the inflatable bulb to be screwed on and manually inflated. It is also cost-prohibitive at about $362 per unit.

One-handed and rapid application is desirable in emergency tourniquet application. Emergency tourniquets typically include a tri-glide loop and Velcro® mechanism. Some prior art describes a ratchet mechanism that must be advanced by the user each time tightening is desired. Although beneficial for precise tightening, this mechanism does not allow for pressure to be monitored and makes periodic reperfusion very difficult without risking ischemic reperfusion injury, as slow release is not described. Additionally, pressure needs change throughout the application of a tourniquet. For example, many patients lose consciousness after massive hemorrhage, causing the limb to relax and bleeding to resume following tourniquet application, which requires additional tightness not monitored or alerted by the windlass tourniquet.

The above-described deficiencies of today's systems are merely intended to provide an overview of some of the problems of conventional systems, and are not intended to be exhaustive. Other problems with the state of the art and corresponding benefits of some of the various non-limiting embodiments may become further apparent upon review of the following detailed description.

In view of the foregoing, it is desirable to provide a system that allows one or few having minimal experience in emergency medical treatment to effectively apply tourniquet(s) to one or more injured individuals in a minimal amount of time with the minimum necessary resources and components.

Thus, there is a need for an intuitive, automated emergency pneumatic tourniquet that eliminates the need for training, helps to prevent tourniquet induced damages, and can be used on limbs of varying sizes.

There is also a need for an automated emergency pneumatic tourniquet that prevents or reduces blood loss and automatically adjusts the pressure of the device based on the width of the limb.

### BRIEF SUMMARY OF THE INVENTION

Disclosed is an automated emergency pneumatic tourniquet system including multiple interchangeable tourniquet cuffs, each having an inflatable bladder and an indicator tag designating the type of cuff, preferred pressure of application, and other data. A detachable controller reads the indicator tag and inflates the bladder to a predetermined pressure, which can be modified once applied. The detachable controller may be used to inflate multiple cuffs individually and monitor the amount the pressure applied by the cuffs. It also allows for periodic reperfusion for reducing and/or preventing ischemia. The cuff is held in place on a limb using a manual tightening mechanism independent of the bladder and controller.

In one embodiment, an automated emergency pneumatic tourniquet system includes a cuff having an inner inflatable bladder, an indicator tag, a connector extending upward from the attachment platform having a check valve and providing fluid communication to the inflatable bladder. The system also includes a tightening mechanism configured to manually attach the cuff to a hemorrhaging limb, and a detachable controller having a PCB, a power supply, a socket configured to detachably connect to the connector of the cuff, a receiver configured to read data stored on the indicator tag of the cuff, a pressure gauge and an inflator both in fluid communication with the socket, and at least one of one or more buttons and a speaker, wherein the controller provides audial and/or visual instructions to an operator of the automated emergency pneumatic tourniquet system.

In one method of using the automated emergency pneumatic tourniquet system, the cuff is placed on a hemorrhaging limb at a location medial and proximal to a point of injury. The operator can power on the controller prior to use, and the controller will provide interactive instructions. The detachable controller is either pre-attached to the cuff or attached after cuff placement on a limb by engaging the connector with the socket such that the controller reads the indicator tag of the cuff. The cuff is secured to the limb by engaging the tightening mechanism. The controller inflates the inflatable bladder to within a pressure range according to the data on the indicator tag. Next, the controller queries the operator to indicate whether the hemorrhaging limb has stopped bleeding. If the operator indicates that the bleeding has not stopped, the controller increases pressure in the inflatable bladder by 10 to 30 mmHg. When the operator indicates that bleeding has stopped, the controller maintains current pressure within the inflatable bladder.

The operator can adjust the pressure of the inflatable bladder. The method can be expanded and used on multiple cuffs by detaching the controller from the cuff, attaching it to a second cuff and repeating the above steps until all of the additional cuffs have been applied to injured limbs. The controller can record the elapsed time and pressure applied for each of the plurality of cuffs and can optionally communicate this data to an external computer. The entire system, including several cuffs and controllers can be stored in a kit in a publicly accessible location.

The controller may additionally provide automatic, periodic reperfusion to the patient's limb, and can also include an electronically stored library of indicator tag data corresponding to preset pressure and reperfusion parameters.

The controller includes the ability for an operator to preset specifications desired for tourniquet application. For example, a trained user may want to bypass interactive instructions and inflate manually. In a setting where covertness is important, such as, but not limited to, a mass shooting, audible instructions and alarms can be silenced in favor of visual instructions. Alternatively, a user anticipating self-application or application to a specific person can input their biometric data, such as blood pressure, heart rate, and more, allowing the controller to calculate their personalized limb occlusion pressure rather than using indicator tag or limb circumference data.

In some embodiments, the tightening mechanism is a ratchet lock. The controller may also optionally include a pulse sensor, and the controller is programmed to cease inflating the bladder once the pulse sensor ceases to detect a pulse.

In other embodiments, the cuff includes an antibiotic delivery mechanism. This delivery mechanism can include a sterile water store, a powdered antibiotic store, a mixer that opens the sterile water store and the powdered antibiotic store to produce an aqueous antibiotic solution, and one or more microneedles through which the aqueous antibiotic solution is delivered into a muscle of the patient wearing the automated pneumatic tourniquet. Alternatively, this system may include a pain management medication. The microneedles can access the muscle and therefore deliver intramuscular antibiotics through the dispersion of adipose tissue from tourniquet pressure. Moreover, releasing the tourniquet to apply less pressure would allow for an intradermal or subcutaneous medication delivery. The microneedles can also be replaced with a cannula for antibiotic or pain medication delivery in some embodiments.

It is therefore an object of the present invention to provide a system to minimize blood loss and prevent death from exsanguinating hemorrhage. It is also an object of the invention to provide tourniquets that are easily correctly applied and adjusted to avoid tourniquet-induced damage to a limb. It is also an object of the invention to provide an automated emergency pneumatic tourniquet that eliminates the need for training, helps to prevent tourniquet induced damages, and can be used on limbs of varying sizes.

These and other objects and advantages of the present invention will become apparent from a reading of the attached specification and appended claims. There has thus been outlined, rather broadly, the more important features of the invention in order that the detailed description thereof that follows may be better understood, and in order that the present contribution to the art may be better appreciated. There are features of the invention that will be described hereinafter and which will form the subject matter of the claims appended hereto.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a perspective view of an automated emergency pneumatic tourniquet system in accordance with principles of the invention;

FIG. 2 is a perspective view of a cuff and tightening mechanism for an automated emergency pneumatic tourniquet system in accordance with principles of the invention;

FIG. 3 is an enlarged perspective view of the tightening mechanism for an automated emergency pneumatic tourniquet system in accordance with principles of the invention;

FIG. 4 is a top perspective view of a cuff and a disengaged tightening mechanism for an automated emergency pneumatic tourniquet system in accordance with the principles of the invention;

FIG. 5 is a side view of a cuff and an engaged tightening mechanism for an automated emergency pneumatic tourniquet system in accordance with the principles of the invention;

FIG. 6 is another side view of a cuff and an engaged tightening mechanism for an automated emergency pneumatic tourniquet system in accordance with principles of the invention;

FIG. 7 is a bottom plan view of a cuffs and a disengaged tightening mechanism for an automated emergency pneumatic tourniquet system in accordance with the principles of the invention;

FIG. 8 is a top perspective view of a controller for an automated emergency pneumatic tourniquet system in accordance with principles of the invention;

FIG. 9 is a bottom perspective view of a controller for an automated emergency pneumatic tourniquet system in accordance with the principles of the invention;

FIG. 10 is a perspective cutaway view of a controller for an automated emergency pneumatic tourniquet system in accordance with the principles of the invention;

FIG. 11 is a cross-sectional view of a controller for an automated emergency pneumatic tourniquet system in accordance with the principles of the invention;

FIG. 12 is a top plan view of a controller being attached to a cuff of an automated emergency pneumatic tourniquet system in accordance with the principles of the invention;

FIG. 13 is a side perspective view of a controller being attached to a cuff of an automated emergency pneumatic tourniquet system in accordance with the principles of the invention;

FIG. 14 is an environmental view of an automated emergency pneumatic tourniquet system affixed to a limb in accordance with the principles of the invention;

FIG. 15 is a cross-sectional view of an automated emergency pneumatic tourniquet system affixed to a limb in accordance with principles of the invention;

FIG. 16 shows a flowchart for a method of using an automated emergency pneumatic tourniquet system in accordance with the principles of the invention;

FIG. 17 shows a flowchart for an alternative method of using an automated emergency pneumatic tourniquet system in accordance with principles of the invention;

FIG. 18 shows a visual instruction for display on a controller for an automated emergency pneumatic tourniquet system in accordance with the principles of the invention;

FIG. 19 shows another visual instruction for display on a controller for an automated emergency pneumatic tourniquet system in accordance with the principles of the invention;

FIG. 20 shows another visual instruction for display on a controller for an automated emergency pneumatic tourniquet system in accordance with the principles of the invention;

FIG. 21 shows another visual instruction for display on a controller for an automated emergency pneumatic tourniquet system in accordance with the principles of the invention;

FIG. 22 shows another visual instruction for display on a controller for an automated emergency pneumatic tourniquet system in accordance with the principles of the invention;

FIG. 23 shows another visual instruction for display on a controller for an automated emergency pneumatic tourniquet system in accordance with the principles of the invention;

FIG. 24 shows another visual instruction for display on a controller for an automated emergency pneumatic tourniquet system in accordance with the principles of the invention;

FIG. 25 shows another visual instruction for display on a controller for an automated emergency pneumatic tourniquet system in accordance with the principles of the invention;

FIG. 26 shows another visual instruction for display on a controller for an automated emergency pneumatic tourniquet system in accordance with the principles of the invention;

FIG. 27 shows another visual instruction for display on a controller for an automated emergency pneumatic tourniquet system in accordance with the principles of the invention;

FIG. 28 shows another visual instruction for display on a controller for an automated emergency pneumatic tourniquet system in accordance with the principles of the invention;

FIG. 29 shows another visual instruction for display on a controller for an automated emergency pneumatic tourniquet system in accordance with the principles of the invention;

FIG. 30 shows another visual instruction for display on a controller for an automated emergency pneumatic tourniquet system in accordance with the principles of the invention;

FIG. 31 shows a perspective view of an alternative embodiment of an automated emergency pneumatic tourniquet system in accordance with principles of the invention;

FIG. 32 shows a perspective view of an alternative embodiment of a cuff for an automated emergency pneumatic tourniquet system in accordance with principles of the invention;

FIG. 33 shows a perspective view of an alternative embodiment of a controller for an automated emergency pneumatic tourniquet system in accordance with principles of the invention;

FIG. 34 shows a poster for displaying visual instruction for an automated emergency pneumatic tourniquet system in accordance with the principles of the invention;

FIG. 35 shows a perspective view of an alternative embodiment of a cuff and an engaged tightening mechanism of an automated emergency pneumatic tourniquet system in accordance with principles of the invention;

FIG. 36 shows another perspective view of an alternative embodiment of an automated emergency pneumatic tourniquet system in accordance with the principles of the invention;

FIG. 37 is an enlarged bottom perspective view of the antibiotic delivery mechanism of an automated emergency pneumatic tourniquet system, in accordance with the principles of the invention;

FIG. 38 is an enlarged top perspective view of the antibiotic reconstitution and delivery system for an automated emergency pneumatic tourniquet system, in accordance with the principles of the invention;

FIG. 39 is a top perspective view of the valve and water reservoir of the antibiotic reconstitution and delivery system for an automated emergency pneumatic tourniquet system, in accordance with the principles of the invention;

FIG. 40 is a perspective view of the pump of the antibiotic reconstitution and delivery system for an automated pneumatic emergency tourniquet, in accordance with the principles of the invention;

FIG. 41 is an perspective view of the containment units of the antibiotic reconstitution and delivery system for an automated emergency pneumatic tourniquet system, in accordance with the principles of the invention;

FIG. 42 is a side elevation view of the containment units and micro-needles of the antibiotic reconstitution and delivery system for an automated emergency pneumatic tourniquet system, in accordance with the principles of the invention;

FIG. 43 is a side elevation view of the gateways of the antibiotic reconstitution and delivery system for an automated emergency pneumatic tourniquet system, in accordance with the principles of the invention;

FIG. 44 is a cross-sectional top view of the containment units of the antibiotic reconstitution and delivery system for an automated emergency pneumatic tourniquet system, in accordance with the principles of the invention;

FIG. 45 is an exploded perspective view of the containment units of the antibiotic reconstitution and delivery system for an automated emergency pneumatic tourniquet system, in accordance with the principles of the invention;

FIG. 46 is another side elevation view of the containment units and micro-needles of the antibiotic reconstitution and delivery system for an automated emergency pneumatic tourniquet system, in accordance with the principles of the invention;

FIG. 47 is a perspective view of another alternative embodiment of an automated emergency pneumatic tourniquet for use with an antibiotic delivery mechanism in accordance with the principles of the invention.

### DETAILED DESCRIPTION

The invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

The disclosed subject matter is described with reference to the drawings, wherein like reference numerals are used to refer to like elements throughout. In the following description, for purposes of explanation, numerous specific details are set forth in order to provide a thorough understanding of the various embodiments of the subject disclosure. It may be evident, however, that the disclosed subject matter may be practiced without these specific details. In other instances, well-known structures and devices may be shown in block diagram form in order to facilitate describing the various embodiments herein.

Various embodiments of the disclosure could also include permutations of the various elements as if each dependent claim was a multiple dependent claim incorporating the limitations of each of the preceding dependent claims as well as the independent claims. Unless explicitly stated otherwise, such permutations are expressly within the scope of this disclosure. Similarly, the disclosure should be interpreted as including permutations of the various elements disclosed in the Figures, unless the various elements are clearly mutually exclusive.

Unless otherwise indicated, all numbers expressing quantities of ingredients, dimensions, reaction conditions and so forth used in the specification and claims are to be understood as being modified in all instances by the term “about”. The term “a” or “an” as used herein means “at least one” unless specified otherwise. In this specification and the claims, the use of the singular includes the plural unless specifically stated otherwise. In addition, use of “or” means “and/or” unless stated otherwise. Moreover, the use of the term “including”, as well as other forms, such as “includes” and “included”, is not limiting. Also, terms such as “element” or “component” encompass both elements and components comprising one unit and elements and components that comprise more than one unit unless specifically stated otherwise.

In the description of the embodiments of the present invention, unless otherwise specified, azimuth or positional relationships indicated by terms such as “up”, “down”, “left”, “right”, “inside”, “outside”, “front”, “back”, “head”, “tail” and so on, are azimuth or positional relationships based on the drawings, which are only to facilitate description of the embodiments of the present invention and simplify the description, but not to indicate or imply that the devices or components must have a specific azimuth, or be constructed or operated in the specific azimuth, which thus cannot be understood as a limitation to the embodiments of the present invention. Furthermore, terms such as “first”, “second”, “third” and so on are only used for descriptive purposes, and cannot be construed as indicating or implying relative importance.

Furthermore, unless otherwise clearly defined and limited, terms such as “installed”, “coupled”, “connected” should be broadly interpreted, for example, it may be fixedly connected, or may be detachably connected, or integrally connected; it may be mechanically connected, or may be electrically connected; it may be directly connected, or may be indirectly connected via an intermediate medium. As used herein, the terms “about” or “approximately” apply to all numeric values, whether or not explicitly indicated. These terms generally refer to a range of numbers that one skill in the art would consider equivalent to the recited values (i.e., having the same function or result). In many instances these terms may include numbers that are rounded to the nearest significant figure. To the extent that the inventive disclosure relies on or uses software or computer implemented embodiments, the terms “program,” “software application,” and the like as used herein, are defined as a sequence of instructions designed for execution on a computer system. A “program,” “computer program,” or “software application” may include a subroutine, a function, a procedure, an object method, an object implementation, an executable application, an applet, a servlet, a source code, an object code, a shared library/dynamic load library and/or other sequence of instructions designed for execution on a computer system. Those skilled in the art can understand the specific meanings of the abovementioned terms in the embodiments of the present invention according to the specific circumstances.

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

Disclosed is an automated emergency pneumatic tourniquet system that includes multiple interchangeable tourniquet cuffs which are programmed to a certain pressure and that can be modified once applied. An electronic controller removably attaches to a cuff, provides instructions to an operator, monitors the pressure applied by the cuff, and allows for periodic reperfusion for reducing and/or preventing ischemia. The tourniquet system is compact and can be stored in public places, and can be integrated into first aid kits such as “stop the bleed” kits.

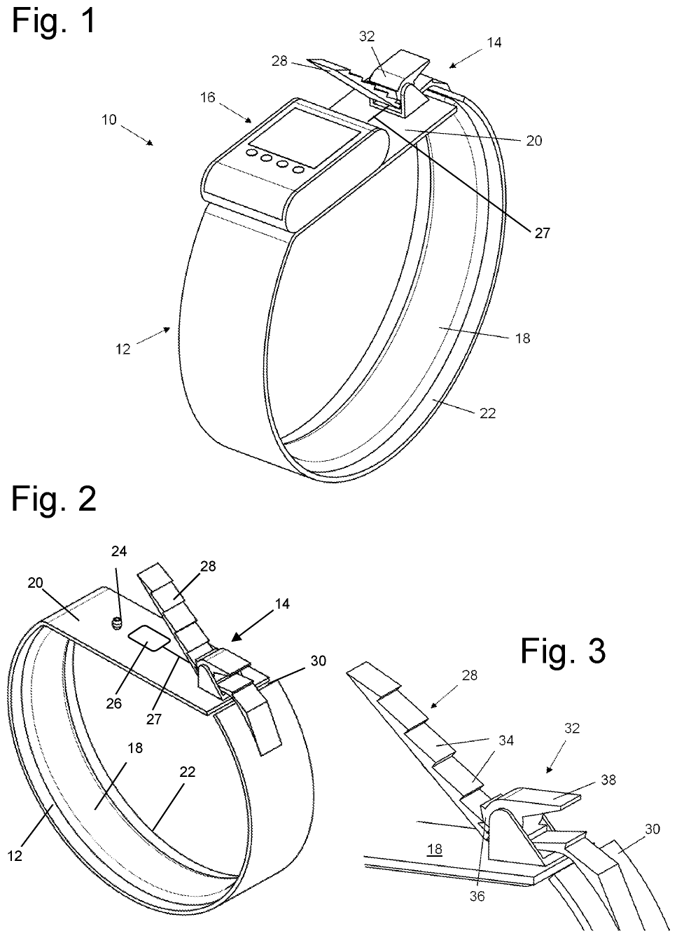

FIGS. 1-15 show an exemplary automated emergency pneumatic tourniquet 10, in accordance with the principles of the invention. The tourniquet 10 includes a cuff 12, a tightening mechanism 14, and a detachable electronic controller 16. In this embodiment, the cuff 12 is sized for a limb circumference of about 23.5 cm to about 31.9 cm and is approximately 11 cm wide and 95.25 cm long. However, the cuff 12 may optionally be sized for either pediatric, regular, obese and/or other types of patients and its width may vary from about 3 cm to 13 cm. Table 1 lists exemplary suitable sizes for different applications:

TABLE 1
Cuff Sizes and Specifications

| Specification | Pediatric | | Regular Adult | | XL Adult | |
|---|---|---|---|---|---|---|
| Limb Circumference | 13 23.4 | cm | 23.5-31.9 | cm | 32-119 | cm |
| Range | | | | | | |
| Width | 3.85 | cm | 11 | cm | 13 | cm |
| Full length | 29.2 | cm | 95.25 | cm | 95.25 | cm |

The cuff 16, as shown in FIGS. 2-7, also includes an interior inflatable bladder 18 and optionally an attachment platform 20. The bladder 18 extends along the inner side 22 of the cuff 12. In this embodiment, the bladder 18 extends substantially the entire length of the inner side 22 of the cuff 12. Optionally, the bladder 18 may extend only partially along the length of the underside 22. However, it is generally preferred to have the bladder 18 extend across most of or all of the inner side 22, as this ensures that pressure is applied equally around a limb to which the tourniquet 10 is attached.

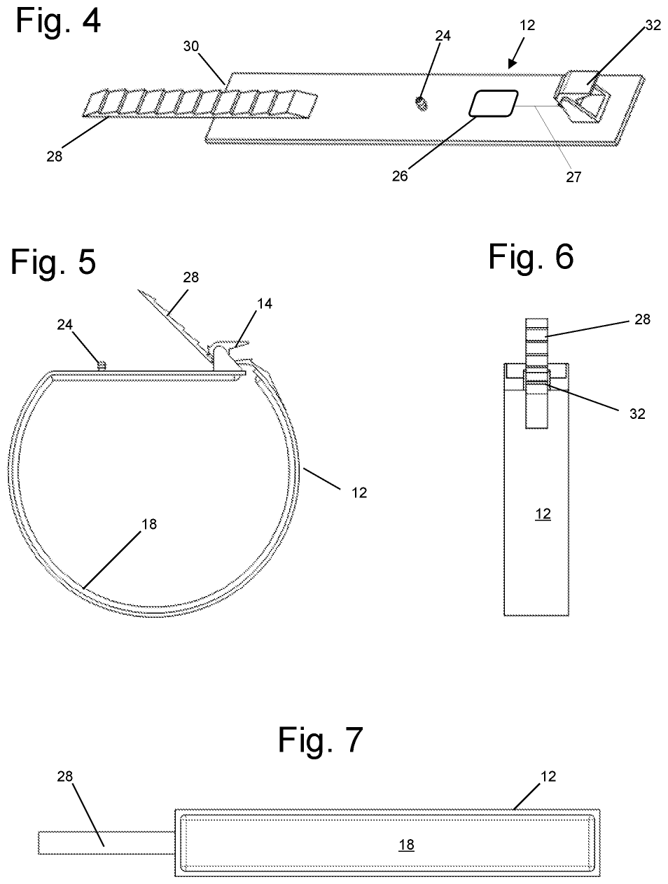

The attachment platform 20 includes a connector 24 and an indicator tag 26 and may be a separate rigid components or may optionally be only a region of the cuff 12. In this embodiment, the attachment platform 20 refers to a region of the cuff 12 opposite to the first end 30 and is defined as the region that includes the connector 24, tag 26 and lock 32. The attachment platform 20 may also include an integrated sensor in the lock 32 connected to the indicator tag 26 embedded within the attachment platform 20. The internal sensor records the length of the amount of the tightening strip drawn through the lock. For example, in this embodiment the sensor tracks the number of teeth 34 pulled through the tightening mechanism 14. This information can be transmitted to the chip and can be used by the controller to calculate the actual circumference of the limb to determine the optimal pressure to be applied by the bladder. Optionally, a flex sensor may be imbedded in the cuff 12 and run along the entire periphery of the cuff 12 to measure the flexion of the cuff, and thus the circumference of the limb. Those skilled in the art will appreciate that there are additional mechanisms that may be integrated into the cuff of the invention to measure the actual length of the limb. The connector 24 is in fluid communication with the bladder 18 and includes an integral check valve. In this embodiment, the connector 24 has a barbed fitting. Optionally, the connector 24 may be a Luer lock, bayonet lock, or the like. The indicator tag 26, shown in FIGS. 2 and 4, interacts with a receiver on the bottom side of the controller 16 as explained in more detail below. In this embodiment, the indicator tag 26 is a passive RFID tag. However, the tag 26 may optionally have a different configuration, such as protrusions or indentions in a predetermined pattern which are readable by the controller 16 or may have a color pattern or bar code readable by the receiver on the controller 16. The indicator tag 26 may provide a variety of information relating to the cuff 12 to the controller 16, via the receiver. For example, the indicator tag 26 may indicate the type and size of the cuff, a singular identification number or code, and recommended pressure ranges for its bladder. Indicator tags can alternatively be programmed to inflate to a personalized limb occlusion pressure preset by the user based on their blood pressure and other biometric indicators. Optionally, the cuff may include a flex sensor to obtain a more accurate cuff size for a limb on which it is affixed.

Referring to FIG. 3, the tightening mechanism 14 of this exemplary embodiment includes a strap 28 extending from the first end 30 of the cuff 12 and is removably insertable into the ratchet lock 32. The strap 28 includes a plurality of ratchet teeth 34. A pawl 36 engages the ratchet teeth 34 to lock the strap 28 to hold the tourniquet 10 in place. The strap 28 may only be removed from the ratchet lock 32 by actuating the release handle 38. In this embodiment, the strap 28 has a width smaller than the transverse width of the cuff 12. Those having ordinary skill in the art will appreciate that other types of tightening and/or locking mechanism such as, but not limited to, a hook and loop strap, a magnetic strap, a buckle, or the like, may be used. Unlike tourniquets found in the prior art, the tightening mechanism 14 of this embodiment is not the primary means of adjusting the tourniquet. The strap and locking mechanism used herein are a means of securing the cuff 12 onto the limb, and the bladder 14 is used as the primary means of adjustment.

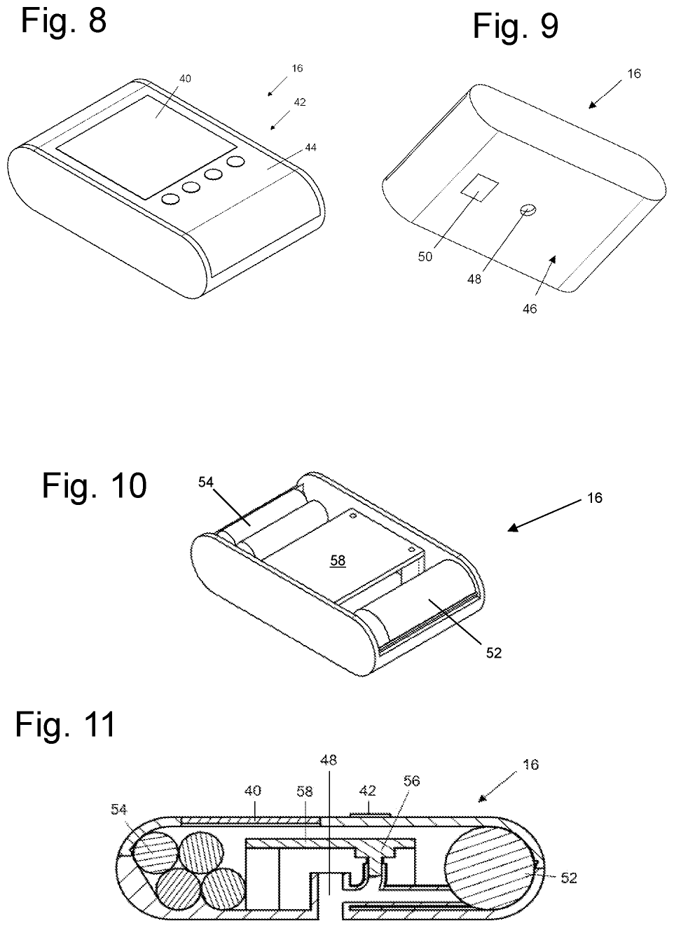

Referring to FIGS. 8-11, the electronic controller 16 includes a display screen 40 and a plurality of buttons 42 on its top side 44. The bottom side 46 includes a socket 48 complementary to the connector 24 and a receiver 50. The electronic controller 16 houses an inflator 52, a power supply 54, a pressure gauge 56 and a processor 58 in communication with an output device 40, an inpute device 42, and a pressure gauge 56. In this embodiment, the output device is a viewscreen, and the input device is a series of buttons 42 along one side of the viewscreen such the images on the viewscreen may be used to identify which buttons correspond to various input signals provided by the operator in response to queries from the output device. One or more buttons may optionally be designated as a power button. The input device is used to turn on, program and otherwise interact with the controller. In this embodiment, there are four buttons 42 arranged linearly along one side of the viewscreen 40 so that the viewscreen may be used to indicate the effect of actuating the various buttons during use of the tourniquet system. This is explained in more detail below. Optionally, as explained below, the output device can include a speaker, and the input device may include a microphone. Optionally, the output and input devices may be combined to a single unit, such as for example a touchscreen capable of displaying images and detecting when virtual buttons on it have been depressed. Those skilled in the art will appreciate that these alternative components are interchangeable and not limited to the specific embodiments shown, unless it is otherwise indicated by the context of this description.

When the socket 48 is attached to the connector 24, the pressure gauge 56 measures the air pressure of bladder 18. The inflator 52 inflates and/or deflates the bladder 18 and may include an air pump and/or a compressed gas, e.g. a CO₂, cartridge. The receiver 50 scans the indicator tag and identifies the type of cuff to which it has been attached, as well as any other information provided by the indicator tag. The socket 48 is configured to engage the check valve in the connector 24 to allow the controller to deflate, as well as inflate, the bladder 18.

The power supply 54 of this embodiment includes a plurality of replaceable batteries. Those skilled in the art will appreciate that there are a nimiety of power supplies available for electronic controllers such as the one shown here. Is generally desirable to utilize a power supply that is highly portable, for example a battery, and the controller 16 of the present invention may also optionally include a micro USB or other socket to provide connection to alternative power supplies, for example an electrical outlet. The controller may also optionally include a plurality of other components such as for example a wireless transmitter, a GPS or other locator module, a thermometer for measuring the temperature of a person to which a cuff is been applied, a speaker, a microphone, one or more LED or other illumination devices, and the like. Those skilled in the art will appreciate that a speaker may be used to convey messages in place of and/or in conjunction with the viewscreen. Similarly, a microphone may be used to receive input signals from an operator in addition to or in place of actuation of the buttons 42. User preferences can be preset prior to use, and audible instructions can be muted or raised if desired during use. Furthermore, the controller may optionally exclude the viewscreen and may be connected instead via Bluetooth to an independent display or mobile device.

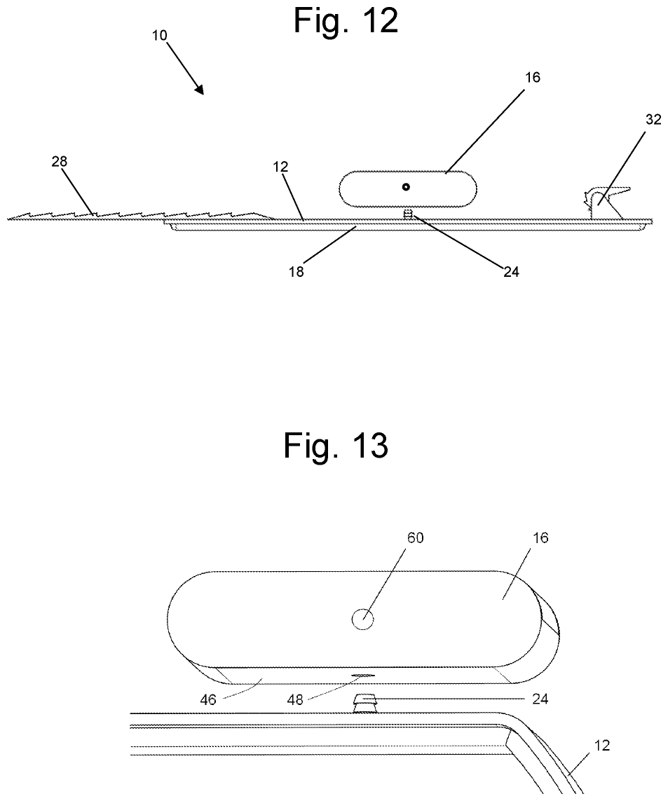

As shown in FIGS. 12 and 13, the electronic controller 16 is removably attached to the attachment platform 20 of the cuff 12 by snapping the socket 48 onto the connector 24. To release the detachable electronic controller 16 from the cuff 12, the button 60 on the side of the electronic controller 16 is depressed. This disconnects the connector 24 from the socket 48 and releases the controller 16. Those skilled in the art will appreciate that the button 60 can alternatively be a switch, slide switch, or other release mechanisms known in the art.

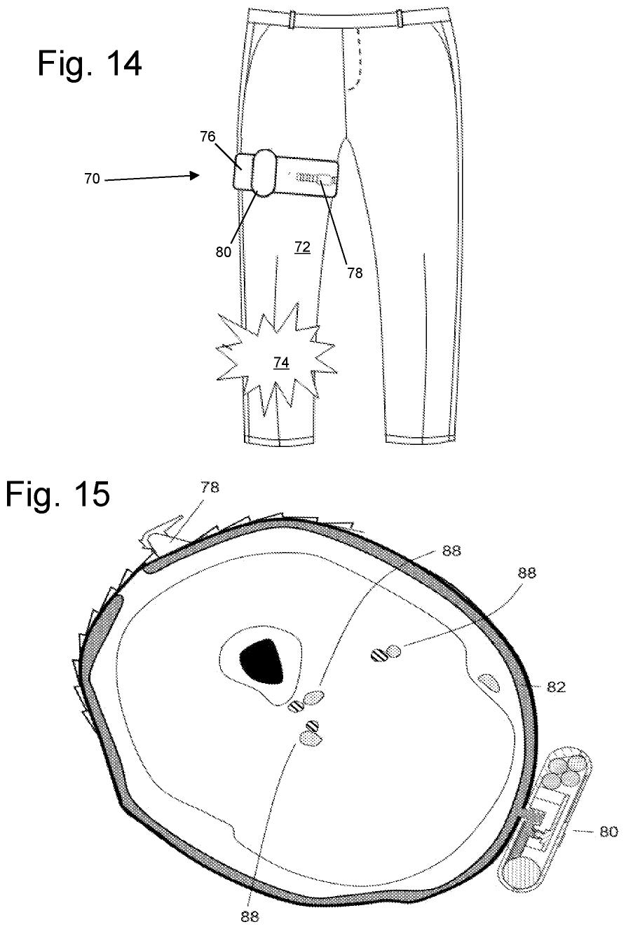

FIGS. 14 and 15 show an automated emergency pneumatic tourniquet 70 of the present invention as used to prevent and/or stop exsanguination from a wound. In one method of use, the tourniquet 70 is placed around a limb 72 at a location medial to a wound 74. An operator fixes the tourniquet 70 to the limb 72 by wrapping the cuff 76 around the limb and manually engaging the tightening mechanism 78. The tightening mechanism 78 creates a sufficient friction fit with the limb to hold the cuff 76 in the proper location. The electronic controller 80 is then attached to the cuff 76. The operator then turns on controller 80, which reads the tag on the cuff 76 and automatically inflates the bladder 82 to a predetermined suitable pressure based upon the information provided by the tag. The controller asks the user to confirm that bleeding has stopped, and then adjusts if necessary. This allows almost anyone, even those with no medical training, to optimize the pressure applied by the tourniquet 70.

Once the bladder 82 is inflated, the controller 80 may be detached. The check valve in the connector of the cuff prevents air from leaking from the bladder 82 and maintains constant pressure. The controller 80 may then be attached to a separate cuff on the same or different patient. Once applied to a subsequent cuff, the controller 80 then reads the tag on that cuff and again inflates the bladder to the desired pressure. The pressure is generally sufficient to cease blood flow through the veins and arteries 88 within the limb 72. This process may be repeated numerous times such that a single controller allows one unskilled person to quickly and rapidly apply several tourniquets to several individuals. The controller 80 may be subsequently reapplied to cuffs that have already been inflated. In this second application of the controller, an operator may verify that a particular cuff is maintaining the correct pressure and not leaking. If the indicator tags on the various cuffs provide singular identifications, the controller can also track the length of time each cuff has been applied. The controller can also record the pressure of various cuffs as the controller is reattached to verify that the correct pressure is being maintained.

The controller may also be used to perform reperfusion on one or more cuffs. If the controller remains on a single cuff, it may periodically emit an audio or visual alarm, for example a beep or a flash, to attract the attention of the operator. The controller is either preset to automatically conduct reperfusion, for example, for a single patient such as in military applications where PC scenarios are likely, or preset to then ask if the operator wishes to conduct a reperfusion using the tourniquet. If an operator indicates yes, then the controller will partially deflate and then reinflate the bladder of the cuff to which it is attached. If the controller has been used on a plurality of cuffs, it may suggest performing reperfusion at regular intervals for some or all of the one or more of the cuffs it has been used to control and is currently tracking the status of.

In one method of use, the electronics unit is pre-attached to the tourniquet. The user will place the tourniquet proximal to the point of injury. An LCD viewscreen provides interactive instructions. The user powers the device “ON” using the buttons on the controller. The device identifies which cuff is attached via an RFID chip and reader, while displaying placement instructions if this setting has not been overridden by a trained user, and the viewscreen indicates that inflation has begun. Once inflated to a preset pressure (according to the RFID chip indicator tag or algorithm based on flex sensor data), the user is asked if bleeding has stopped. If “YES” is indicated on buttons, the tourniquet maintains pressure, and the screen displays the elapsed time and pressure. After a period of time has passed, which varies according to the pressure applied, the screen asks if the user would like to reperfuse the limb. If “YES” is indicated, the electronics unit slowly releases air over a period of time to maximize perfusion but prevent exsanguination. This will allow for maximum tissue oxygenation and prevent the probability of both ischemic-reperfusion injury and nerve damage following tourniquet application.

The tourniquet can also be replaced by a hemostatic dressing after a set period of time as is standard procedure in literature and fielded traumatic wound care. The user will be notified of this after time elapses and guided through the process of packing the wound, applying pressure, checking for additional hemorrhage, and deciding whether or not to leave the tourniquet on. Reperfusion cycles can occur in preset increments for the entire tourniquet application. The instructions recommend that removal occurs under medical supervision. When removal is desired, the user depresses the release switch on the tightening mechanism, which may be protected by a safety in some embodiments to prevent the accidental release of the cuff.

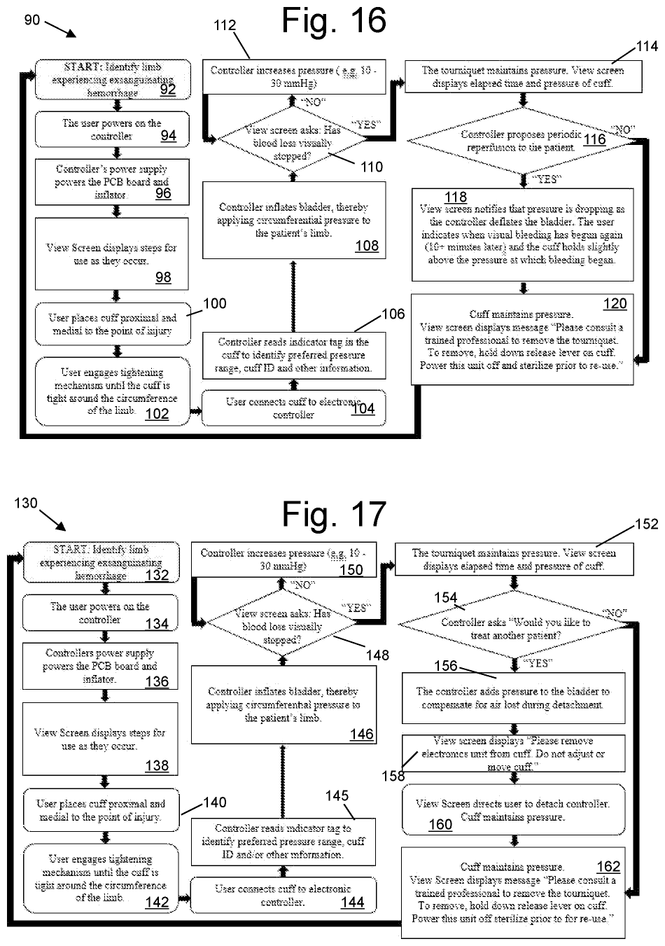

FIG. 16 shows a flowchart 90 of a method of using the automated emergency pneumatic tourniquet for a single injury in accordance with principles of the invention. The method begins by an operator identifying a limb that is experiencing an exsanguinating hemorrhage 92. Once a limb in need of attention is identified, the user powers on the controller 94. The controller's power supply then provides power to the internal electrical components 96, such as the processor board and inflator. To assist an unskilled operator, the controller displays a series of instructions on its view screen 98, as explained in more detail below. The user then places a cuff on the limb medial to the exsanguinating hemorrhage 102. The user then connects the cuff to the controller 104. In step 106, the controller scans and reads the indicator tags on the cuff. Based on the information provided by the tag, the controller pressurizes the bladder 108. Once the bladder 108 is pressurized, the controller, via the viewscreen for a speaker, asks the operator if the bleeding appears to have stopped. The operator responds by audibly stating “yes” or “no” or by depressing a “yes” or “no” button or selecting “yes” or “no” on a touchscreen, as explained below. If the operator responds with a “no,” the controller increases pressure by a predetermined amount, for example 10 to 30 mmHg., in step 112. If an operator responds with a “yes,” then the controller maintains the current pressure. In step 116, after a predetermined amount of time, for example five minutes, the controller asks the operator, via the viewscreen or a speaker, whether it should perform reperfusion. If the operator answers in the negative, then the controller maintains the current pressure and may optionally continue to query whether reperfusion should be performed every so often, e.g., every five minutes. If the operator answers in the affirmative, then in step 118 the controller notifies the operator, via the viewscreen or a speaker, that it is slowly decreasing pressure. When an operator observes that visual bleeding has begun, which may occur ten or more minutes after the reduction in pressure, he or she notifies the controller by pressuring a button or stating a verbal command. Upon receiving this notification from an operator, the controller increases the pressure slightly, for example about 10-30 mmHg, and maintains that pressure. The cuff maintains the pressure and the viewscreen recommends to the user to seek medical attention prior to removing the tourniquet 120. Alternatively, the reperfusion metrics can be preset by the operator to only release a certain amount over a set time interval. The viewscreen may also provide the user with instructions on how to remove the controller. Optionally, the controller may periodically lower and raise pressure in order to provide microcirculatory blood flow to reduce ischemia therefore to prevent ischemia reperfusion injury distal to the tourniquet. Alternatively, the controller can be pre-programmed to release a small amount of pressure, for example 5-100 mmHg.

FIG. 17 shows another exemplary method 130 for using the automated emergency pneumatic tourniquet system in accordance with the principles of the invention. The method described in FIG. 17 is suitable for situations in which a single controller is used with several separate, independent cuffs. As with the previous method, it begins with step 132, identifying a limb experiencing exsanguinating hemorrhage. An operator turns on the controller in step 134, providing power to the PCB board, inflator, and any other components 136 of the controller. Again, in 138 the view screen displays the steps of the method to assist an operator. An operator, or user, in step 140 places a cuff proximal and medial to a point of injury. In step 142, the user engages the tightening mechanism to generate a friction fit of the cuff around the limb. The user then, in step 144 connects the cuff to the electronic controller. In both of the above methods, the controller can be connected to the cuff before engaging the tightening mechanism, however, it is generally easier to perform the steps in the order given here. Once attached to the cuff, the receiver on the controller reads the indicator tags on the cuff and inflates the cuffs bladder accordingly 145. The controller then inflates the bladder, applying pressure to the patient's limb 146. When the predetermined pressure is reached, the controller asks the user if the bleeding has stopped 148. If the bleeding has not stopped, then the controller proceeds to step 150 and increases pressure, for example 10-30 mmHg, and again asks if the bleeding has stopped. This process repeats until the user indicates that the bleeding has stopped. At that point, in step 152, the controller maintains the current pressure. Alternatively, those experienced with the automated tourniquet (i.e., a first responder) can override this adjustment step by manually increasing or decreasing the pressure using buttons as described below. The controller also tracks the amount of time over which the tourniquet has been applied. The controller also asks the operator in step 154 if there are additional cuffs requiring adjustment by the controller. If not, then the controller simply maintains the current pressure, step 162, unless the operator directs otherwise. If instead there are additional cuffs to be adjusted, then the controller slightly increases pressure on the current cuff to compensate for any air pressure lost when the controller is detached 156. The viewscreen then directs the user to remove the controller from the cuff without moving or adjusting the cuff 158. The check valve in the cuff maintains constant pressure after the controller has been removed 160. Once the controller has been removed, it may be attached to another cuff on another limb of the same patient or on a separate patient. If the indicator tags on the various cuffs include individual identification data, then the controller can keep track of the amount of time each individual cuff has been engaged. The controller can also indicate to a user whether it might be desirable to perform reperfusion on any of the cuffs. The controller may also optionally remind the operator to reconnect the controller to the various cuffs to verify that the correct pressure is being maintained. The controller may also optionally transmit data regarding the pressure applied and duration of pressure application for each cuff to a mobile device or computer network. For example, the data collected by the controller may be readily transferred to medical professionals and/or uploaded to a patient's files.

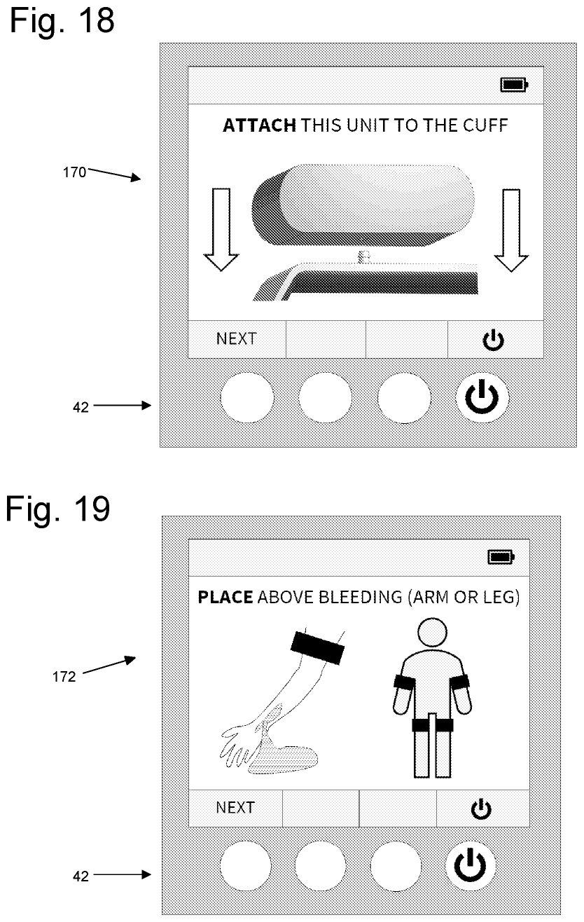

FIGS. 18 to 30 show exemplary instructions that may be displayed on the viewscreen 40 of the controller 16 shown in FIGS. 8-11. Below, the different instructions have been corresponded to steps in flowchart 90 shown in FIG. 16. Those skilled in the art will appreciate that these instructions also similarly correspond to similar or identical steps of flowchart 130 and FIG. 17. They may optionally be accompanied by audio instructions provided by a speaker such as the one described in more detail in an alternative embodiment below. FIG. 18 shows an initial instruction 170. These instructions 170 and include both an image and a brief written instruction to attach the controller to the cuff by snapping the socket onto the connector. This corresponds to step 104 of flowchart 90 shown in FIG. 16. The viewscreen 40 also includes an arrow pointing to one of the buttons 42 or a symbol or a word corresponding to one of the buttons 42 which is to be depressed after the step is completed. If a controller includes a touch screen, as described below, then the buttons may simply be part of the image displayed on the screen. When the “next” button is depressed, the instructions 172 of FIG. 19 replace instructions 170. Instructions 170 direct an operator to place the cuff, or “strap,” in the correct position, i.e., medial to an injury. This step corresponds approximately to step 100 and flowchart 90 of FIG. 16. Instructions 172 also indicate which button to depressed when the step is completed.

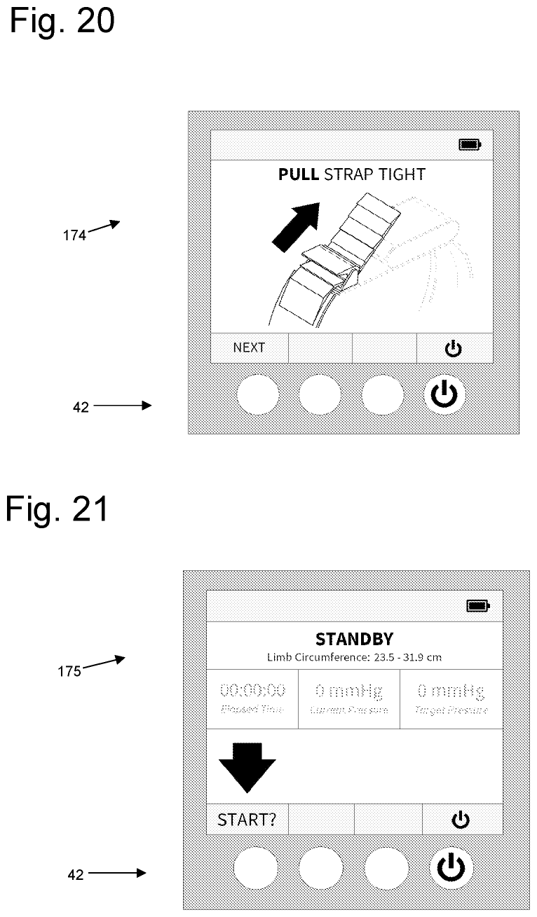

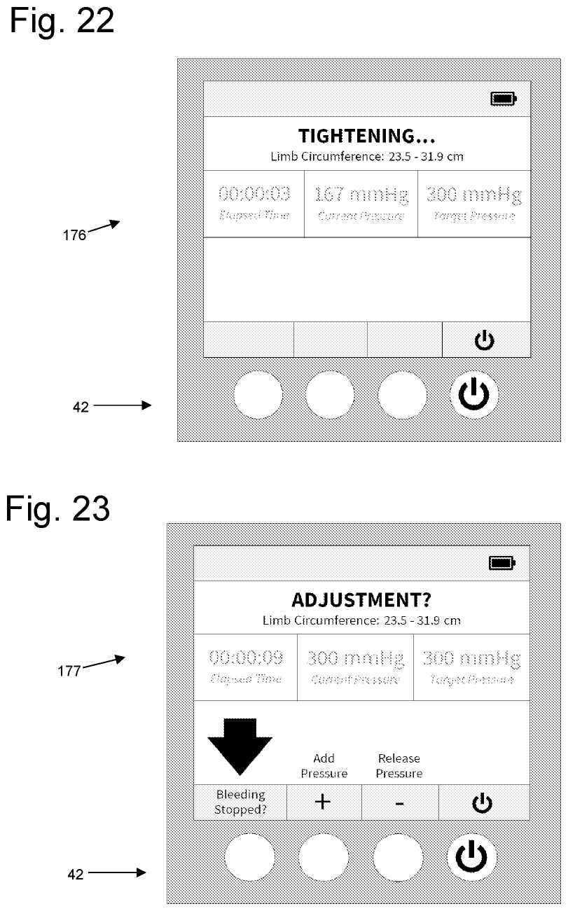

FIG. 20 shows the next instructions 174, both showing and describing to the user that the tightening mechanism is to be engaged to secure the cuff about a limb. This corresponds to step 102 in flowchart 90 shown in FIG. 16. As described above, the order of the steps can be rearranged as demonstrated by the sequence of the instructions shown in FIGS. 18-30. Once again, the instructions 174 also identify a “next” button. When the next button is depressed, instructions 175 shown in FIG. 21 is displayed. This “Standby” image indicates that the controller is attached to the cuff, detected the size of the cuff, in this example “Limb Circumference 23.5-31.9 cm,” and is ready to begin inflating the bladder. When the “start” button is depressed, the controller begins inflating the bladder, and the screen displays instructions 176 shown in FIG. 22, indicating that the bladder is inflating.

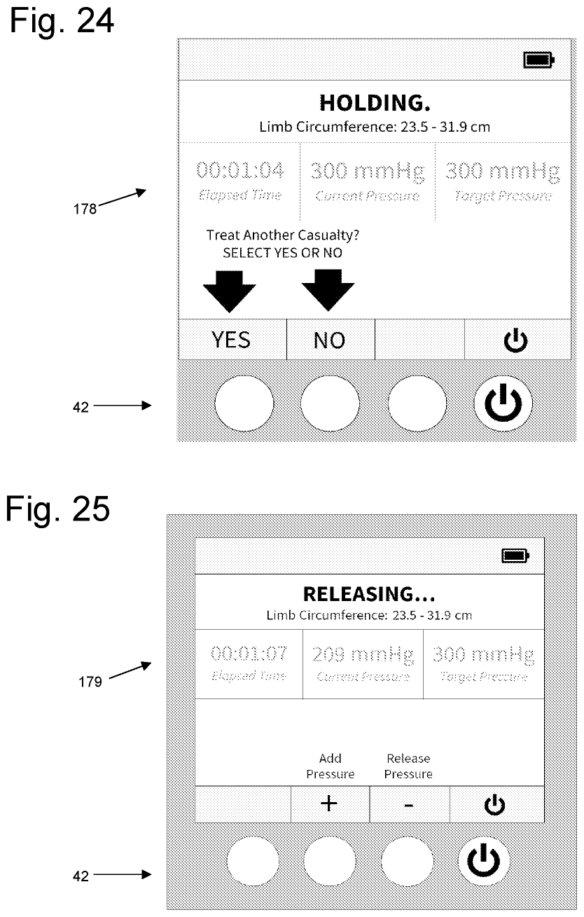

Once the bladder has been inflated by the controller to the predetermined pressure, depending on the type of cuff to which the controller is attached, the instructions 177 in FIG. 23 are displayed. These instructions ask the user whether bleeding has stopped, and if the “bleeding stopped” button is pressed, the next instructions shown in FIG. 24 are displayed. If bleeding has not stopped, the user may increase pressure by simply depressing the correct button or may also optionally reduce pressure. This corresponds to the query step 110 in flowchart 90 of FIG. 16. The instructions also display the lapsed time that the cuff is been inflated, a target pressure for the bladder, and the actual pressure within the bladder. This allows an operator or user to confirm that the controller has correctly read the indicator tag or flex sensor data from the cuff to which it is attached. Instructions 178 indicate that the pressure is “holding” at the current level and asks if the user wishes to use the controller to adjust another cuff.

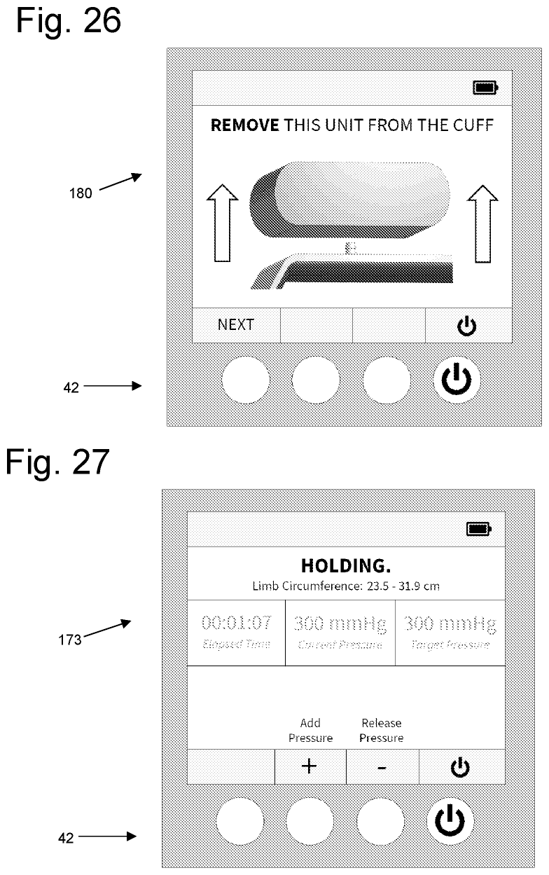

If the user wishes to treat another patient, the “yes” button is depressed. If this button is depressed, the image shown in FIG. 25 is displayed, indicating that the controller is disconnecting itself from the cuff, leaving the cuff at the pressure displayed. Next, the viewscreen will display instructions 180 shown in FIG. 26. These instructions simply direct the user to remove the controller from the cuff. The precise method of removing the controller will vary depending on the type of connector and socket used to join the two components. And additional instructions may optionally be provided to ensure that the controller is properly detached. The controller may then be attached to another cuff, and the controller repeats the same instructions and images on the subsequent cuff. This process can be repeated as many times as necessary.

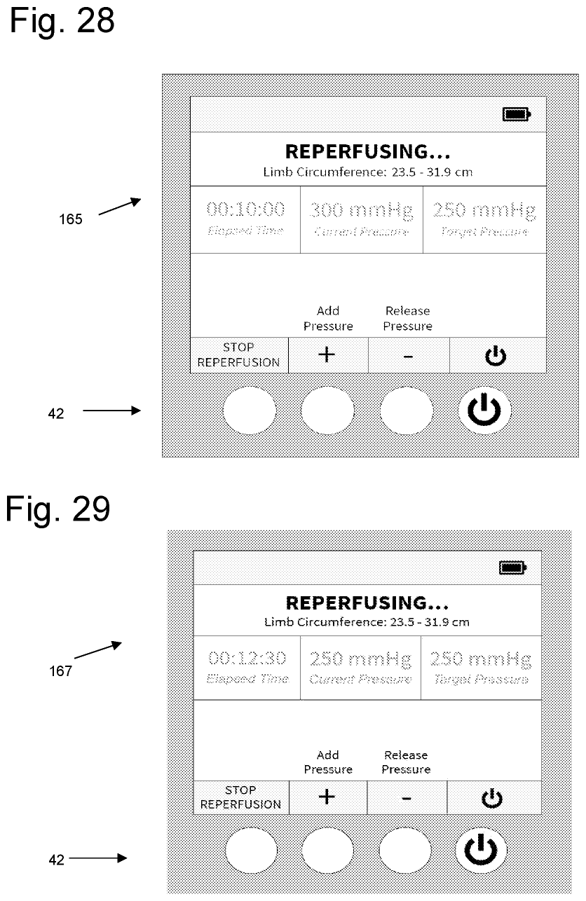

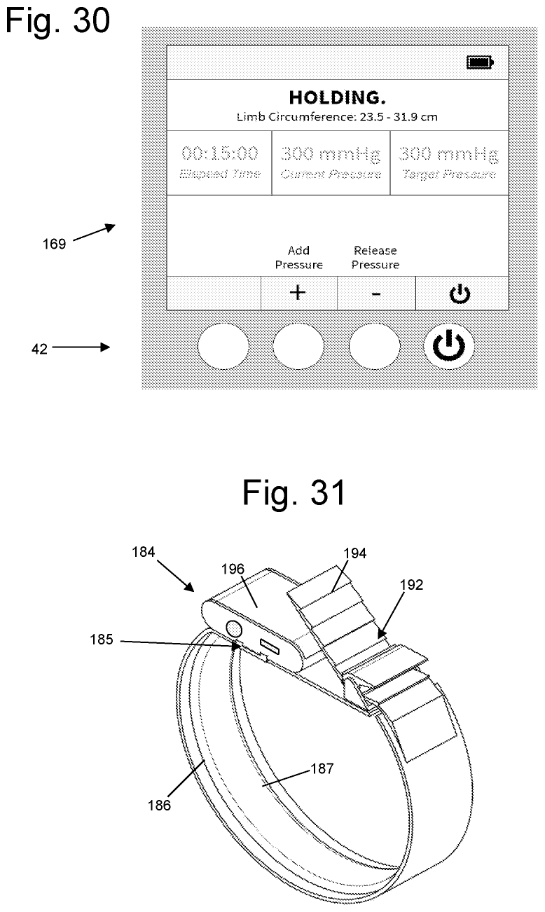

FIGS. 27-30 show the case in which the user selected “no” to the visual instructions 178 presented in FIG. 23. The controller may then present the display instructions 173 in FIG. 27. This indicates that the cuff is holding pressure and gives an operator or user the option to increase or decrease pressure. FIG. 28 displays the visual instructions 165 present during periodic reperfusion of the cuff. During reperfusion, the viewscreen may present the elapsed time, the current pressure, and the target pressure. The viewscreen also gives the user the option to stop reperfusion, add pressure, or release pressure during the reperfusion. FIG. 29 displays the instructions 167 on the viewscreen once the targeted pressure has been reached. FIG. 30 displays the visual instructions 169 on the viewscreen once reperfusion has been complete. The instructions 169 also display the lapsed time that the cuff is been inflated, a target pressure for the bladder, and the actual pressure within the bladder. These steps corresponds to the steps 116, 118, and 120 in flowchart 90 of FIG. 16. Laypeople may not understand the concept of reperfusion, so to prevent confusion once the unit is first set up it can explain the need for reperfusion to the user and give them an opportunity to automatically set reperfusion up without asking during emergency application.

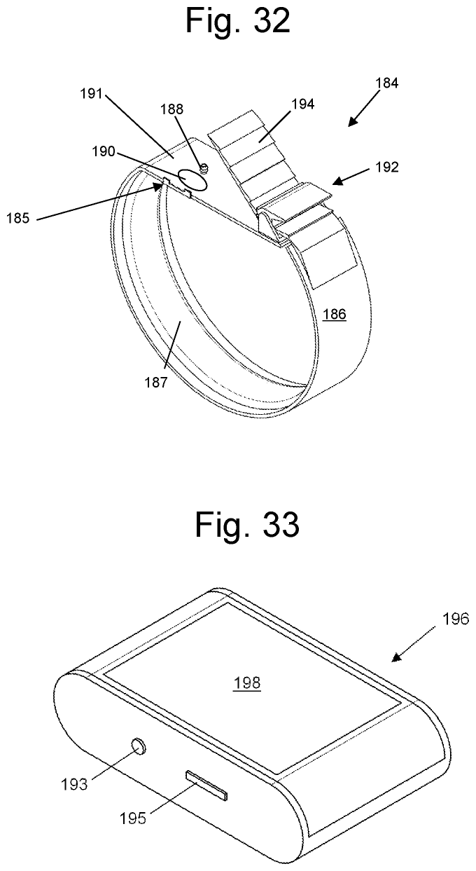

FIGS. 31-34 show an alternative embodiment of an automated emergency pneumatic tourniquet 184 in accordance with the principles of the invention. The cuff 186 is substantially similar to the cuff 12 shown in FIGS. 4-7, with only slight modifications. In this embodiment, the cuff 186 includes an inflatable bladder 187 on its inner side, a connector 188 configured as a Luer lock, and the indicator tag 190 is positioned to one side of it. In this embodiment, the cuff 186 further includes opposing alignment brackets 185 on each side of the attachment platform 191. The alignment brackets 185 ensure that the receiver of the controller 196 is aligned with the indicator tag 190. While brackets 185 are used in this embodiment, those having ordinary skill in the art would appreciate that other alignment mechanisms are also suitable for properly aligning the controller 196 in the correct orientation and/or prevent undesired rotation of the controller 196 about the connector 188 from occurring.

The tightening mechanism 192 uses the same type of ratchet lock as tightening mechanism 14, but the tightening strap 194 is wider. The controller 196 includes a touch screen 198, and therefore does not include buttons for interacting with the controller 196. The controller 196 also includes a speaker 193 and a microphone 195. This allows the controller 196 to issue audio instructions in addition to or in place of visual instructions. The microphone 195 allows the controller 196 to receive audial responses to its queries, so that the user may simply answer “yes” or “no” in response to questions. The controller 196 may also be configured to recognize specific questions.

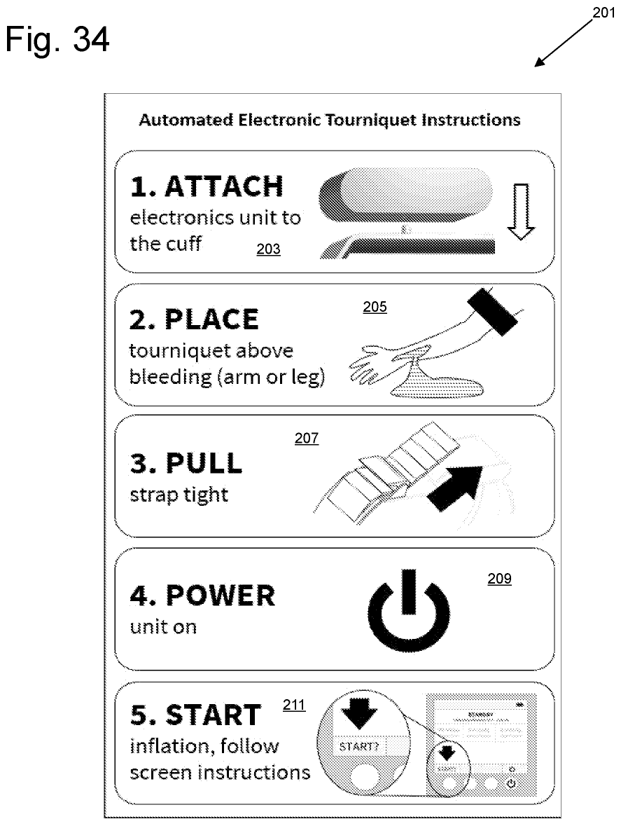

FIG. 34 displays a poster 201 that may be temporarily attached to a wall in a public location, such as, but not limited to, a school, adjacent to one or more cuffs and one or more controllers. The poster 201 walks the user through the first few steps of applying the automated emergency pneumatic tourniquet 184 to a patient. The first step 203 directs the user to attach the controller to the cuff. The poster 201 then directs the user to place the tourniquet above the bleeding limb 205. The next step directs the user to pull the strap tight 207, and power on the controller 209. Finally, the poster 201 directs the user to begin inflation and follow the instructions present on the viewscreen 211. Though the instructions in this embodiment are printed on a poster, the same step by step instruction may instead be made into a sticker or may even be printed or stitched on the outside of the cuff.

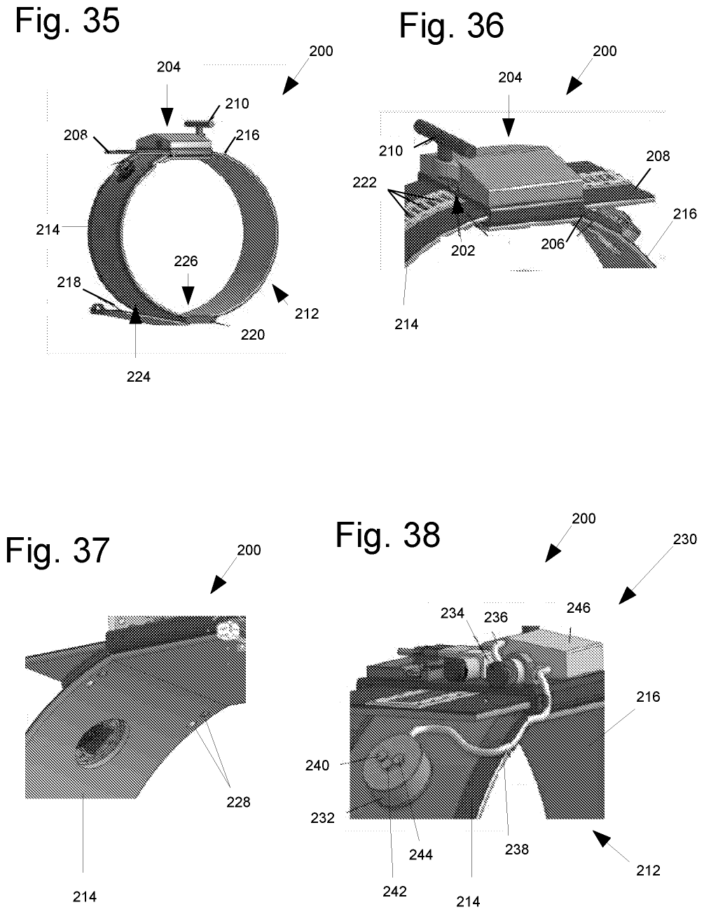

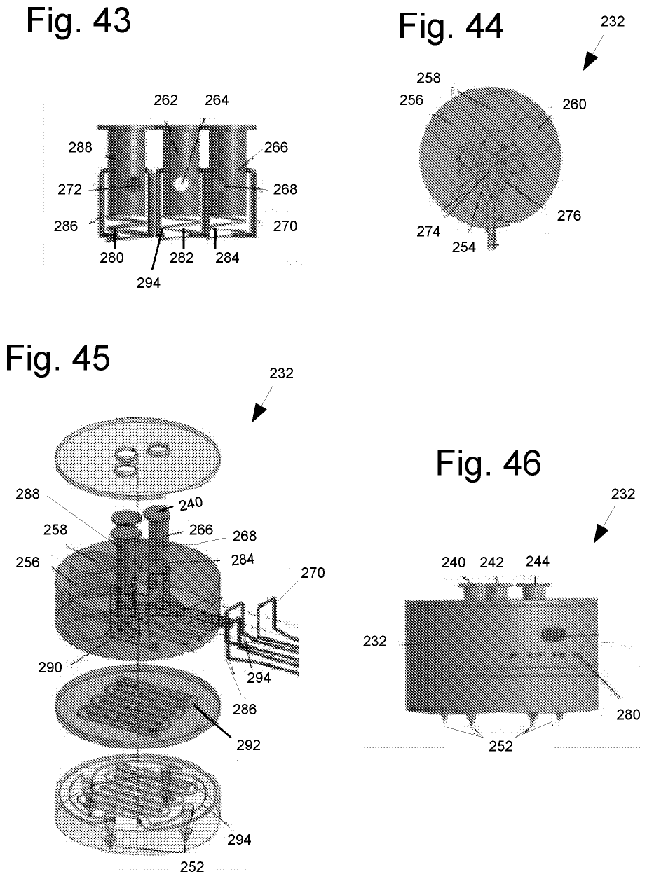

FIGS. 35-46 show an alternative embodiment of an automated emergency pneumatic tourniquet 200, in accordance with the principles of the invention. The cuff 212 is comprised of a first strap portion 214 and a second strap portion 216 that are configured to encircle an injured limb. The first and second straps 214 and 216 have distal ends 218 and 220 that meet and join to form the cuff 212. A controller 204 is mounted on a proximal end 206 of the first strap portion 214, and the proximal end 208 of the second strap 216 passes through channel 202 between the controller 204 and the first strap 214. The proximal end 208 of the second strap 216 includes a drive engagement feature that includes a series of transverse slots 222 that engage with the threads of a worm gear in the controller 204. Thus, the controller 204 draws the proximal end 208 of the second strap 216 through the channel 202 to tighten the automated emergency pneumatic tourniquet 200. Likewise, the controller 204 can reverse and allow the tourniquet 200 to loosen. The controller 204 is responsive to pressure sensors 228 that sense a pulse in the affected limb and adjust the pressure until no pulse is detected. The distal ends 218 and 220 of the straps 214 and 216 meet at a clasp 226, and the first strap 214 doubles back on itself, such that the end can be fastened to the middle portion 224 of the first strap by 214, for example, hook and loop fasteners. This allows for an initial adjustment of the automated tourniquet 200. Finally, the tourniquet 200 includes a manual crank 210 that allows for manual tightening of the straps 214 and 216 in the case of mechanical failure of the controller. Once the cuff 212 is fully tightened, either manually or mechanically, antibiotic reconstitution and delivery by the antibiotic delivery mechanism can begin.

FIGS. 37-46 display various detail views of the antibiotic delivery mechanism 230 of the automated emergency pneumatic tourniquet 200. The antibiotic delivery mechanism 230 is designed to administer Ertapenem intramuscularly (IM) 1 gm/day following reconstitution with 4 mL of water but can be easily made compatible with any powdered antibiotic. After the tightening process is complete and the patient's pulse rate is undetected, the reconstitution process can begin. The controller 204 sends a signal to activate the pumping mechanism 232. The signal causes the spring pump 234 to compress. This creates a vacuum in the pump 204 and draws liquid from the reservoir 246. In this embodiment, the reservoir contains a dialysis-type bag holding ˜18.7 mL sterile water as a diluent for reconstitution. However, any liquid suitable for drug reconstitution may fill the reservoir. The pumping mechanism 242 must run eight cycles. A single “cycle” is completed when the spring pump 234 compresses and decompresses once. Eight cycles pulls a total of 4 mL of liquid [(0.5 mL/1 cycle)*(8 cycles)=4 mL] from the reservoir 236 for one IM dose of Ertapenem. Each cycle forces liquid through the one-way valve 230 by pumping water from the reservoir 246, through the tube 236, through port 250, through the valve 230, and through port 248, and into tube 238. The final tube 238 serves as the connection between the pumping system and the reconstitution system, as it delivers liquid from the pumping system into the reconstitution system.

Pre-dosed powdered antibiotics are stored in containment units 256, 258, and 260. In this embodiment, 1 g INVANZ Ertapenem is stored in each unit 256, 258, and 260. Three gateways 266, 262, and 288 within the pumping mechanism 232 prevent liquid from combining with powder until the proper time. At set intervals, one of the three shape memory alloy (SMA) wires 270, 286, and 294 receives a signal from the controller 204. This pulls down the respective gateway 266, 262, and 288 and compresses the respective spring 280, 282, and 284. When the respective spring 280, 282, and 284 is compressed, the gateway 266, 262, and 288 is aligned with the respective hole 264, 268, and 272, permitting the liquid in one of the respective tubes 254, 274, and 276 to flow through the hole 264, 268, and 272 and access the respective containment unit 256, 258, and 260. The powder stored in the containment unit 256, 258, and 260 is reconstituted once liquid diluent flows through the hole 264, 268, and 272 and contacts the powder. Once the powder is reconstituted, the liquid antibiotic travels downward through the 3D Microfluidic structure 290, 292, and 294. The antibiotic is thoroughly mixed by traveling through the first 3D Microchannel 290 using the energy of the pumping mechanism 232. It then wraps through the other two microchannels 292 and 294, where it is finally pushed through the plurality of microneedles 252, which administer the antibiotics into the patient's muscle. This process repeats for each of the three loaded Ertapenem doses and will be programmed to administer automatically 1 g/IM/24 h. Alternatively, it can be programmed to administer the antibiotic at a different pressure to access different parts of the body, for example intradermal or subcutaneous tissue.

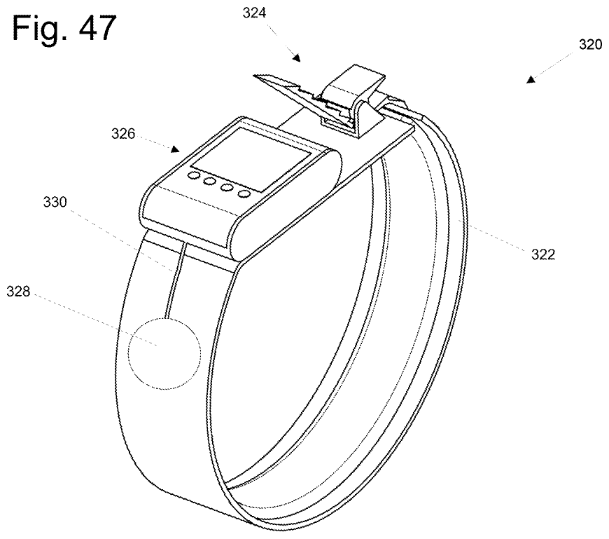

The automated pneumatic emergency tourniquet delivery system is designed such that the antibiotic reconstitution and administration system have a manual option as a fail-safe or by user preference. To operate the pumping mechanism, the user will push and pull the spring pump 234 back and forth for eight cycles, while holding down one of the gate's corresponding buttons 240, 242, and 244. Holding down one of the buttons 240, 242, and 244 will manually align the holes 264, 268, and 272, therefore allowing reconstitution and administration. If the manual option is selected, and if the tourniquet is being used in a prolonged care scenario, the patient must manually time the 24 h period or period described by FDA labeling for Ertapenem antibiotic, but this will vary depending on the antibiotic type. Detailed instructions for this fail safe option will be printed directly onto the device when manufactured in order to increase compliance among injured personnel. Those skilled in the art will appreciate that the microneedles and the antibiotic reconstitution system is not limited to this embodiment alone, and that the method of applying intramuscular antibiotics through tourniquet compression and dispersion of adipose tissue can be incorporated into the prior embodiments, or into other emergency tourniquet systems. Furthermore, a cannula may be used in place of the microneedles, where a constant dose of antibiotics or pain medication is desired. For this application, the tourniquet pressure can be manipulated, with decreased pressure allowing for transdermal, intradermal or subcutaneous drug delivery, and increased pressure dispersing tissue to provide intramuscular access. This antibiotic reconstitution and delivery system is integrated into the pneumatic tightening mechanism in FIG. 47.

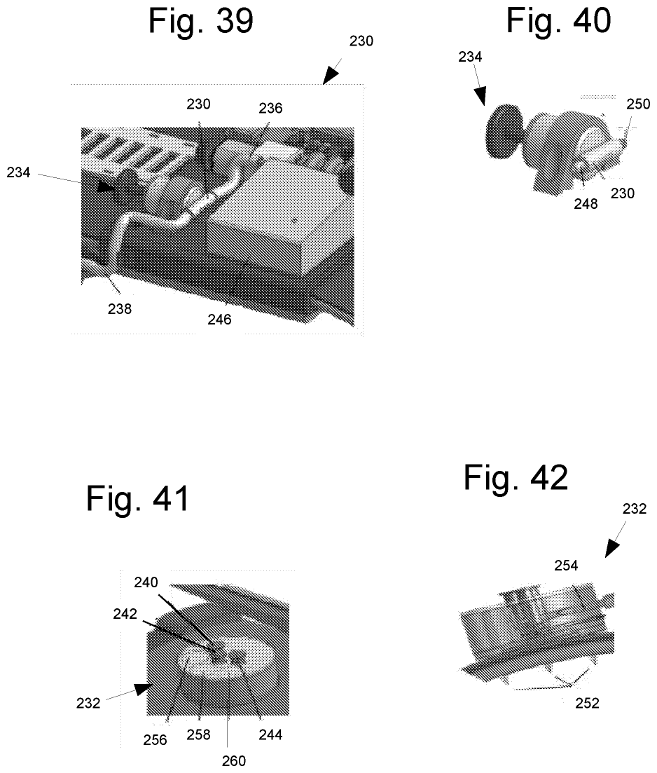

FIG. 47 shows an automated emergency pneumatic tourniquet system 320 having a cuff 322, tightening mechanism 324 and controller 326 very similar to those shown in previous embodiments. However, tourniquet system 320 includes a socket 328 configured to accommodate attachment of a pumping mechanism 232 shown in FIGS. 44-46. Controller 326 is the same as controller 16, except that it has been modified to also house the delivery mechanism components shown in FIGS. 38 and 39 used in conjunction with the pumping mechanism 232, and are in communication with the pumping mechanism 232 via conduit 330. An antibiotic delivery system may be integrated into the system 320, or may be provided in conjunction with it for optional attachment.

The automated emergency pneumatic tourniquet system of the present invention allows a single controller to be utilized with several relatively inexpensive tourniquet cuffs. Such a system is well-suited to serve as a “kit” that may be maintained wherever first aid kits are desirable. Because they require little size, last indefinitely, and are relatively inexpensive and are easy to use, the systems of the present invention are well-suited for use in schools, public transportation, cars, boats, camping, military applications, and the like. They allow persons with little or no medical knowledge to effectively reduce or eliminate exsanguination for one or several people in an efficient manner that minimizes ischemia and other tourniquet related damage. In addition, the system of the present invention provides effective, automated reperfusion without the need for a user with medical training in some embodiments.

Whereas the present invention has been described in relation to the drawings attached hereto, other and further modifications, apart from those shown or suggested herein, may be made within the spirit and scope of this invention. Those skilled in the art will appreciate that the conception, upon which this disclosure is based, may readily be utilized as a basis for the designing of other structures, methods, and systems for carrying out the several purposes of the present invention. That is, the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting. The descriptions of the embodiments shown in the drawings should not be construed as limiting or defining the ordinary and plain meanings of the terms of the claims unless such is explicitly indicated. The claims should be regarded as including such equivalent constructions insofar as they do not depart from the spirit and scope of the present invention.

1. An automated emergency pneumatic tourniquet system for treating a hemorrhaging limb with comprising:
At least one cuff having an attachment platform, an inner inflatable bladder, an indicator tag, and a connector extending upward from the attachment platform, wherein the connector comprises a check valve and provides fluid communication to the inflatable bladder;
a tightening mechanism on the at least one cuff configured to manually attach the at least one cuff to a hemorrhaging limb; and
a detachable controller having a processor, a power supply, a socket configured to detachably connect to the connector of the cuff, a receiver configured to read data stored on the indicator tag of the cuff, a pressure gauge, an inflator in fluid communication with the socket, an output device, and at least one input device, wherein the controller provides stepwise instructions to an operator of the automated emergency pneumatic tourniquet system.

2. The automated emergency pneumatic tourniquet system for treating a hemorrhaging limb of claim 1 wherein the controller records the elapsed time and pressure applied by the at least one cuff.

3. The automated emergency pneumatic tourniquet system for treating a hemorrhaging limb of claim 2 wherein the controller includes an electronically stored library of indicator tags and corresponding preset pressure and reperfusion parameters.

4. The automated emergency pneumatic tourniquet system for treating a hemorrhaging limb of claim 3 wherein the controller provides automatic, periodic reperfusion to the patient's limb.

5. The automated emergency pneumatic tourniquet system for treating a hemorrhaging limb of claim 4 wherein the output device is a viewscreen, and the stepwise instructions include images displayed on the viewscreen.

6. The automated emergency pneumatic tourniquet system for treating a hemorrhaging limb of claim 5 wherein the attachment platform further comprises an alignment bracket which only allows the controller to be attached to the attachment platform in an orientation that aligns the indicator tag with the receiver.

7. The automated emergency pneumatic tourniquet system for treating a hemorrhaging limb of claim 6 wherein the at least one cuff comprises a plurality of cuffs.

8. The automated emergency pneumatic tourniquet system for treating a hemorrhaging limb of claim 7 wherein the cuff further comprises an antibiotic delivery mechanism that is actuated by the controller comprising:
a sterile water store;
a powdered antibiotic store;
a mixer that opens the sterile water store and the powdered antibiotic store to produce an aqueous antibiotic solution;
one or more microneedles through which the aqueous antibiotic solution is delivered into a muscle on the limb of the patient to which the automated emergency pneumatic tourniquet system is attached.

9. A method of treating a hemorrhaging limb with an automated emergency pneumatic tourniquet system comprising the steps of:
a) providing an automated pneumatic emergency tourniquet comprising:
a cuff having an attachment platform, an inner inflatable bladder, an indicator tag, and a connector extending upward from the attachment platform, wherein the connector comprises a check valve and provides fluid communication to the inflatable bladder;
a tightening mechanism on the cuff configured to manually attach the cuff to a hemorrhaging limb;
a detachable controller having a processor, a power supply, a socket configured to detachably connect to the connector of the cuff, a receiver configured to read data stored on the indicator tag of the cuff, a pressure gauge, an inflator in fluid communication with the socket, a viewscreen, and an input device, an electronic storage module storing an executable program for displaying a plurality stepwise instructions wherein the controller is capable of providing visual instructions on the viewscreen to an operator of the automated emergency pneumatic tourniquet system;
b) placing the cuff on a hemorrhaging limb at a location medial to a point of injury;
c) securing the cuff to the limb by engaging the tightening mechanism;
d) attaching the detachable controller to the cuff by engaging the connector with the socket such that the controller reads the indicator tag of the cuff;
e) the operator powering on the controller;
f) the controller displaying stepwise instructions on the viewscreen;
g) the controller inflating the inflatable bladder to within a pressure range according to the data on the indicator tag;
h) the viewscreen on the controller querying the operator to indicate whether the hemorrhaging limb has stopped bleeding;
i) the controller increasing the pressure in the inflatable bladder by 10 to 30 mmHg, when the operator indicates that the bleeding has not stopped, and repeats steps g and h;
j) the controller maintaining the current pressure within the inflatable bladder, when the operator indicates that bleeding has stopped.

10. The method of treating a hemorrhaging limb with an automated emergency pneumatic tourniquet system of claim 9 further comprising the steps of:
k) providing a plurality of additional cuffs for treating of a plurality of additional injuries;
l) providing instructions for detaching the controller from the cuff;
m) attaching the controller to a second cuff and repeating steps b-m until all of the plurality of additional cuffs have been applied to injured limbs.

11. The method of treating a hemorrhaging limb with an automated emergency pneumatic tourniquet system of claim 10 wherein the operator can adjust the pressure of the inflatable bladder.

12. The method of treating a hemorrhaging limb with an automated emergency pneumatic tourniquet system of claim 11 wherein the controller records the elapsed time and pressure applied for each of the plurality of cuffs.

13. The method of treating a hemorrhaging limb with an automated emergency pneumatic tourniquet system of claim 12 wherein the plurality of cuffs and the controller are stored in a kit in a publicly accessible location.

14. The method of treating a hemorrhaging limb with an automated emergency pneumatic tourniquet system of claim 13 wherein the controller provides automatic, periodic reperfusion to the patient's limb.

15. The method of treating a hemorrhaging limb with an automated emergency pneumatic tourniquet system of claim 14 wherein the controller includes an electronically stored library of indicator tag data corresponding to preset pressure and reperfusion parameters.

16. The method of treating a hemorrhaging limb with an automated emergency pneumatic tourniquet system of claim 15 wherein the tightening mechanism is a ratchet lock.

17. The method of treating a hemorrhaging limb with an automated emergency pneumatic tourniquet system of claim 16 wherein the controller further comprises a pulse sensor, and wherein the controller is programmed to cease inflating the bladder once the pulse sensor ceases to detect a pulse.

18. The device for treating a hemorrhaging limb with an automated emergency pneumatic tourniquet system of claim 17 wherein the attachment platform further comprises an alignment bracket.

19. The method of treating a hemorrhaging limb with an automated emergency pneumatic tourniquet system of claim 18 wherein the cuff further comprises an antibiotic delivery mechanism that is actuated by the controller comprising:
a sterile water store;
a powdered antibiotic store;
a mixer that opens the sterile water store and the powdered antibiotic store to produce an aqueous antibiotic solution;
one or more microneedles through which the aqueous antibiotic solution is delivered into a muscle on the limb of the patient to which the automated emergency pneumatic tourniquet system is attached.

20. A method of treating a hemorrhaging limb with an automated emergency pneumatic tourniquet system comprising steps executed by a server:
a) providing an automated pneumatic emergency tourniquet comprising:
a cuff having an attachment platform, an inner inflatable bladder, an indicator tag, and a connector extending upward from the attachment platform;
wherein the connector comprises a check valve and provides fluid communication to the inflatable bladder;
a tightening mechanism configured to manually attach the cuff to a hemorrhaging limb;
a detachable controller having a PCB, a power supply, a socket configured to detachably connect to the connector of the cuff, a receiver configured to read data stored on the indicator tag of the cuff, a pressure gauge, an inflator in fluid communication with the socket, a viewscreen, and at least one of one or more buttons;
wherein the controller is capable of providing visual instructions on the viewscreen to an operator of the automated emergency pneumatic tourniquet system;
b) placing the cuff on a hemorrhaging limb at a location medial to a point of injury;
c) securing the cuff to the limb by engaging the tightening mechanism;
d) attaching the detachable controller to the cuff by engaging the connector with the socket such that the controller reads the indicator tag of the cuff;
e) an operator powering on the controller;
f) the viewscreen displaying instructions to the operator;
g) the controller inflating the inflatable bladder to within a pressure range according to the data on the indicator tag;
h) the viewscreen on the controller querying the operator to indicate whether the hemorrhaging limb has stopped bleeding;
i) the controller increasing the pressure in the inflatable bladder by 10 to 30 mmHg, when the operator indicates that the bleeding has not stopped, and repeats steps g and h;
j) the controller maintaining the current pressure within the inflatable bladder, when the operator indicates that bleeding has stopped.
